(19) United States
(12) Patent Application Publication
(10) Pub. No.: US 2007/0244102 A1
(43) Pub. Date: Oct. 18, 2007

(54) Combination of Dermaciclane and Opoids as Analgesics

(76) Inventors: Istvan Gacsalyi, Budapest (HU); Gabor Gigler, Budapest (HU); Laszlo Gabor Harsing, Budapest (HU); Gyorgy Levay, Budakeszi (HU); Gabor Szenasi, Budapest (HU)

Correspondence Address: K.F. ROSS P.C., 5683 RIVERDALE AVENUE, SUITE 203 BOX 900, BRONX, NY, 10471-0900, US

(21) Appl. No.: 10/592,462

(22) PCT Filed: Mar. 12, 2004
(86) PCT No.: PCT/HU04/00021

§ 371 Date: Jun. 14, 2007

(51) Int. Cl. A61K 31/135; A61K 31/13; A61K 31/167; A61K 31/192; A61K 31/215; A61K 31/225; A61K 31/4152; A61K 31/439; A61K 31/542

(52) U.S. Cl. 514/226.5; 514/282; 514/407; 514/533; 514/568; 514/569; 514/629; 514/646; 514/647; 514/658; 514/659

(57) ABSTRACT

The invention relates to a combined analgesic pharmaceutical composition which comprises as component: A) (1R,2S,4R)-(−)-2-[N,N-(dimethylaminoethoxy)]-2-phenyl-1,7-7-trimethylbicyclo[2.2.1]heptane or a pharmaceutically acceptable acid addition salt thereof and as component B) morphine, an opioide type analgesic and/or a non-opioide type analgesic in admixture with suitable pharmaceutical carriers and/or auxiliary agents. Deramciclane increases the analgesic effect of morphine.

### FIELD OF THE INVENTION

The invention relates to a combined analgesic pharmaceutical composition.

### TECHNICAL BACKGROUND

(1R,2S,4R)-(−)-2-[N,N-(dimethylaminoethoxy)]-2-phenyl-1,7,7-trimethylbicyclo[2.2.1]heptane of the Formula

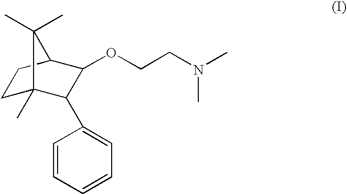

(International Non-Proprietory Name: deramciclane) is an anxiolytic pharmaceutical active ingredient which falls under the general Formula of HU 179,174. The preparation of deramciclane is described in HU 212,574.

Deramciclane showed considerable effects in different animal models of anxiety and stress. In the Vogel punished drinking test deramciclane was active in 1 and 10 mg/kg after oral administration [Gacsályi et. al, Receptor binding profile and anxiolytic activity of deramciclane (EGIS-3886) in animal models, Drug Dev. Res. 40: p. 338-348, (1997)]. In the social interaction model, the compound increased the time spent with social interactions after the single 0.7 mg/kg oral treatment. In the light-dark model, [Crawley, J. N. Neuropharmacological specifity of a simple model of anxiety for the behavioural actions of benzodiazepine, Pharmacol. Biochem. Behavior, 15: p. 695-699 (1981)] deramciclane proved to be active in a single oral dose of 3 mg/kg sc. In the marble burying model [Broekkamp, C. L. et al, Major Tranquilizers Can Be Distinguished from Minor Tranquillisers on the Basis of Effects on Marble Burying and Swim-Induced Grooming in Mice. Eur. J. Pharmacol. 126: p. 223-229, (1986)] the molecule was active in 10 and 30 mg/kg after oral treatment. Deramciclane did not possess analgesic activity in the above mentioned doses.

Additionally to its analgesic potential morphine, unfortunately, has numerous side effects which limit its therapeutic use. These side effects are tolerance, psychic and physiological dependence, and sometimes, fatal depression of respiration.

The object of the present invention is to develop a medical product which potentiates the analgesic effect of morphine.

Further object of the present invention is to develop a pharmaceutical product having analgesic effect.

The above objects are solved by the development of the combined medical product of the present invention.

The invention is based on the recognition that (1R,2S,4R)-(−)-2-[N,N-(dimethylaminoethoxy)]-2-phenyl-1,7,7-trimethylbicyclo[2.2.1]heptane of the Formula I and pharmaceutically acceptable acid addition salts thereof potentiate the analgesic effect of morphine.

Further basis of the present invention is the recognition that the combination of (1R,2S,4R)-(−)-2-[N,N-(dimethylaminoethoxy)]-2-phenyl-1,7,7-trimethylbicyclo[2.2.1]heptane of the Formula I and pharmaceutically acceptable acid addition salts thereof with morphine, opioide type of analgesics and/or other type of analgesics have outstanding analgesic efficacy.

The morphine potentiating effect of deramciclane, from the therapeutical point of view, is very advantageous because by this means the effective dose of morphine can be reduced resulting in the occurrence of less unwanted side effects.

The combination of deramciclane with low doses of morphine is beneficial in the following respects:
1. One will be able to successfully use morphine in low doses in which no effect and consequently no side effects occur (Table 1). In combination with deramciclane, the analgesic effect occurred and has been potentiated without abuse potential (deramciclane has no abuse potential).
2. If one uses the doses of morphine in which the analgesic effects are expressed (e.g. in cancer therapy), deramciclane will further potentiate the analgesic effect of morphine. This means that to achieve the same analgesic effect one can use lower doses of morphine with less side effects.
3. In patients where morphine tolerance has already been developed, in combination with deramciclane the doses of morphine could be decreased and/or not increased further.

The present invention relates to combined pharmaceutical compositions for treating pain and preventing the symptoms of withdrawal from morphine.

### SUMMARY OF THE INVENTION

According to the present invention there is provided a combined analgesic pharmaceutical composition which comprises as component A) (1R,2S,4R)-(−)-2-[N,N-(dimethylaminoethoxy)]-2-phenyl-1,7,7-trimethylbicyclo[2.2.1]heptane or a pharmaceutically acceptable acid addition salt thereof and as component B) morphine, an opioide type analgesic and/or a non-opioide type analgesic in admixture with suitable pharmaceutical carriers and/or auxiliary agents.

### DETAILED DESCRIPTION OF THE INVENTION

The combined analgesic pharmaceutical composition according to the present invention comprises as component A) preferably (1R,2S,4R)-(−)-2-[N,N-(dimethylaminoethoxy)]-2-phenyl-1,7,7-trimethylbicyclo[2.2.1]heptane-2-(E)-butenedioate (1:1).

The combined analgesic pharmaceutical composition according to the present invention comprises as component A) particularly preferably (1R,2S,4R)-(−)-2-[N,N-(dimethylaminoethoxy)]-2-phenyl-1,7,7-trimethylbicyclo[2.2.1]heptane of the Formula I or a pharmaceutically acceptable acid addition salt thereof which contains not more than 0.2% of (1R,3S,4R)-(−)-3-[2-N,N-(dimethylaminoethyl)]-1,7,7-trimethylbicyclo[2.2.1]heptane-2-one of the Formula

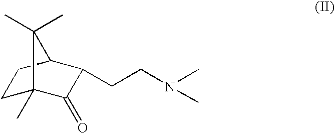

or a pharmaceutically acceptable acid addition salt thereof.

The combined analgesic pharmaceutical composition according to the present invention comprises as component A) particularly preferably (1R,2S,4R)-(−)-2-[N,N-(dimethylaminoethoxy)]-2-phenyl-1,7,7-trimethylbicyclo[2.2.1]heptane-2-(E)-butenedioate (1:1) which contains not more than 0.2% of (1R,3S,4R)-(−)-3-[2-N,N-(dimethylaminoethyl)]-1,7,7-trimethylbicyclo[2.2.1]heptane-2-one-2-(E)-butenedioate (1:1).

(1R,2S,4R)-(−)-2-[N,N-(dimethylaminoethoxy)]-2-phenyl-1,7,7-trimethylbicyclo[2.2.1]heptane of the Formula I or a pharmaceutically acceptable acid addition salt thereof which contains not more than 0.2% of (1R,3S,4R)-(−)-3-[2-N,N-(dimethylaminoethyl)]-1,7,7-trimethylbicyclo[2.2.1]heptane-2-one of the Formula II or a pharmaceutically acceptable acid addition salt thereof is described in Hungarian patent application HU 1559/99.

The combined analgesic pharmaceutical composition according to the present invention comprises as opioide type analgesic preferably tramadol, codeine, dihydrocodeine, nalbufine or buprenorfine or a pharmaceutically acceptable salt thereof.

The combined analgesic pharmaceutical composition according to the present invention comprises as non-opioide type analgesic preferably acetyl salicylic acid, paracetamol, aminophenazone, diclofenac, naproxen, ibuprofene, piroxicam or a pharmaceutically acceptable salt thereof.

The combined analgesic pharmaceutical composition according to the present invention comprises component A) and as component B) morphine or an opioide type analgesic in a weight ratio of 500:1, preferably 100:1, particularly preferably 30:1.

The combined analgesic pharmaceutical composition according to the present invention comprises component A) and as component B) a non-opioide type analgesic in a weight ratio of 1:500, preferably 1:100, particularly preferably 1:30.

The daily dosage of the combined analgesic pharmaceutical composition according to the present invention is generally 0.1-150 mg/kg, preferably 1-150 mg/kg, particularly preferably 10-150 mg/kg.

The term “pharmaceutically acceptable acid addition salt” relates to salts formed with pharmaceutically acceptable inorganic or organic acids. For salt formation e.g. hydrochloric acid, hydrogen bromide, sulfuric acid, phosphoric acid, lactic acid, citric acid, tartaric acid, fumaric acid, maleic acid, succinic acid, benzenesulfonic acid, p-toluenesulfonic acid etc. can be used. (1R,2S,4R)-(−)-2-[N,N-(dimethylaminoethoxy)]-2-phenyl-1,7,7-trimethylbicyclo[2.2.1]heptane of the Formula I can be particularly advantageously used in the form of the fumarate i.e. as (1R,2S,4R)-(−)-2-[N,N-(dimethylaminoethoxy)]-2-phenyl-1,7,7-trimethylbicyclo[2.2.1]heptane-2-(E)-butenedioate (1:1).

The pharmaceutical composition according to the present invention can be prepared in galenic forms generally used in pharmaceutical industry. The compositions may be solid or liquid (e.g. tablets, coated tablets, dragées, capsules, solutions etc.). The pharmaceutical compositions may be administered orally or parenterally, preferably orally. The combined pharmaceutical compositions according to the present invention can be prepared by procedures of pharmaceutical industry known per se.

According to a further aspect of the present invention there is provided a process for the preparation of combined analgesic pharmaceutical compositions which comprises admixing as component A) (1R,2S,4R)-(−)-2-[N,N-(dimethylaminoethoxy)]-2-phenyl-1,7,7-trimethylbicyclo[2.2.1]heptane or a pharmaceutically acceptable acid addition salt thereof and as component B) morphine, an opioide type analgesic and/or a non-opioide type analgesic with inert pharmaceutically acceptable carriers and/or auxiliary agents and bringing the mixture into a galenic form.

According to a still further aspect of the present invention there is provided the use of a combination comprising as component A) (1R,2S,4R)-(−)-2-[N,N-(dimethylaminoethoxy)]-2-phenyl-1,7,7-trimethylbicyclo[2.2.1]heptane or a pharmaceutically acceptable acid addition salt thereof and as component B) morphine, an opioide type analgesic and/or a non-opioide type analgesic for the alleviation of pain.

According to a still further aspect of the present invention there is provided the use of a combination comprising as component A) (1R,2S,4R)-(−)-2-[N,N-(dimethylaminoethoxy)]-2-phenyl-1,7,7-trimethylbicyclo[2.2.1]heptane or a pharmaceutically acceptable acid addition salt thereof and as component B) morphine, an opioide type analgesic and/or a non-opioide type analgesic for the preparation of analgesic pharmaceutical compositions.

According to a still further aspect of the present invention there is provided a process for the alleviation of pain which comprises administering to the patient in need of such treatment a pharmaceutically active dose of a combination comprising as component A) (1R,2S,4R)-(−)-2-[N,N-(dimethylaminoethoxy)]-2-phenyl-1,7,7-trimethylbicyclo[2.2.1]heptane or a pharmaceutically acceptable acid addition salt thereof and as component B) morphine, an opioide type analgesic and/or a non-opioide type analgesic.

According to a still further aspect of the present invention there is provided the use of (1R,2S,4R)-(−)-2-[N,N-(dimethylaminoethoxy)]-2-phenyl-1,7,7-trimethylbicyclo[2.2.1]heptane or a pharmaceutically acceptable acid addition salt thereof to increase the effect of morphine.

The potentiation of the analgesic effect of morphine by deramciclane was demonstrated on hot plate test in mice. A modified of method of Eddy et al. (Eddy, N. B., Leimback, D., Synthetic Analgesics II. Dithienylbutenyl and Dithienylbutylamines. J. Pharmacol. Exp. Ther. 107: p. 385-393, 1953) was used. Male NMRI nice (20-25 g bodyweight) were dropped on a hot plate (56±0.5° C.) and the latency time elapsed until licking the forepaws was measured. The reaction time was tested twice before treatment. Animals were discarded if the first basal latency time>5 sec. or the difference between the two control measurements was greater than 3 sec. Mice were treated either with saline or with morphine HCl 1 mg/kg subcutaneously and at the same time either with vehicle or with deramciclane or buspirone, HCl 30 mg/kg intraperitoneally, respectively. After the treatment (15, 30, 45 and 60 min.) the reaction time was measured again. Animals were regarded as positive if they produced a 2.5-fold reaction time increase at least twice compared to their first control values. Statistical analysis was performed using the chi-square method. The results are shown in Table 1.

TABLE 1
The influences of deramciclane and buspiron on
morphine analgesia in hot plate test in mice

| | Positive/treated | Effect |
|---|---|---|
| Treatment | animals | % |
| Saline sc. + | 0/10 | 0 |
| 0.4% methyl cellulose ip. | | |
| Saline sc. + | 0/10 | 0 |
| Deramciclane - 30 mg/kg ip. | | |
| Morphine HCl - 1 mg/kg sc. + | 0/10 | 0 |
| 0.4% methyl cellulose ip. | | |
| Morphine HCl - 1 mg/kg sc. + | 6/10* | 60 |
| Deramciclane - 30 mg/kg ip. | | |
| Saline sc. + Saline ip. | 0/10 | 0 |
| Saline sc. + | 0/10 | 0 |
| Buspiron HCl - 30 mg/kg ip. | | |
| Morphine HCl - 1 mg/kg sc. + | 0/10 | 0 |
| Saline ip. | | |
| Morphine HCl - 1 mg/kg sc. + | 1/10 | 10 |
| Buspiron HCl - 30 mg/kg ip. | | |

*p < 0.05

Neither morphine HCl 1 mg/kg sc. nor deramciclane 30 mg/kg ip. nor buspiron HCl 30 mg/kg ip. alone produced analgesia in this test. Surprisingly, if the ineffective doses of morphine HCl (1 mg/kg sc.) and deramciclane (30 mg/kg ip.) were administered at the same time then the analgesic effect of this combination was statistically significant. The anxiolytic buspiron did not influence the analgesic effect of morphine.

These results prove that the potentiation of the analgesic effect of morphine by deramciclane is surprising because it does not follow from its anxiolytic effect. This is also emphasized by the experimentally certified recognition according to which neither deramciclane nor buspiron possess analgesic effect in itself.

What we claim is:

1. Combined analgesic pharmaceutical composition which comprises as component A) (1R,2S,4R)-(−)-2-[N,N-(dimethylaminoethoxy)]-2-phenyl-1,7,7-trimethylbicyclo[2.2.1]heptane or a pharmaceutically acceptable acid addition salt thereof and as component B) morphine, an opioide type analgesic and/or a non-opioide type analgesic in admixture with suitable pharmaceutical carriers and/or auxiliary agents.

2. Combined pharmaceutical composition according to claim 1 comprising as component A) (1R,2S,4R)-(−)-2-[N,N-(dimethylaminoethoxy)]-2-phenyl-1,7,7-trimethylbicyclo[2.2.1]heptane-2-(E)-butenedioate (1:1).

3. Combined pharmaceutical composition according to claim 1 comprising as component A) (1R,2S,4R)-(−)-2-[N,N-(dimethylaminoethoxy)]-2-phenyl-1,7,7-trimethylbicyclo[2.2.1]heptane of the Formula 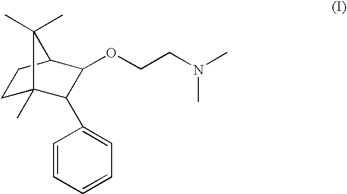
or a pharmaceutically acceptable acid addition salt thereof which contains not more than 0.2% of (1R,3S,4R)-(−)-3-[2-N,N-(dimethylaminoethyl)]-1,7,7-trimethylbicyclo[2.2.1]heptane-2-one of the Formula 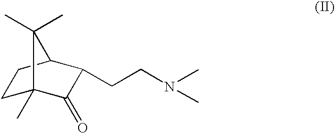
or a pharmaceutically acceptable acid addition salt thereof.

4. Combined pharmaceutical composition according to claim 3 comprising as component A) (1R,2S,4R)-(−)-2-[N,N-(dimethylaminoethoxy)]-2-phenyl-1,7,7-trimethylbicyclo[2.2.1]heptane-2-(E)-butenedioate (1:1) which contains not more than 0.2% of (1R,3S,4R)-(−)-3-[2-N,N-(dimethylaminoethyl)]-1,7,7-trimethylbicyclo[2.2.1]heptane-2-one-2-(E)-butenedioate (1:1).

5. Combined pharmaceutical composition according to any of claims 1-4 comprising as opioide type analgesic [component B)] tramadol, codeine, dihydrocodeine, nalbufine or buprenorfine or a pharmaceutically acceptable salt thereof.

6. Combined pharmaceutical composition according to any of claims 1-4 comprising as non-opioide type analgesic [component B)] acetyl salicylic acid, paracetamol, aminophenazone, diclofenac, naproxen, ibuprofene, piroxicam or a pharmaceutically acceptable salt thereof.

7. Combined pharmaceutical composition according to any of claims 1-6 comprising component A) and as component B) morphine or an opioide type analgesic in a weight ratio of 500:1, preferably 100:1, particularly preferably 30:1.

8. Combined pharmaceutical composition according to any of claims 1-6 comprising component A) and as component B) a non-opioide type analgesic in a weight ratio of 1:500, preferably 1:100, particularly preferably 1:30.

9. Process for the preparation of combined analgesic pharmaceutical compositions which comprises admixing as component A) (1R,2S,4R)-(−)-2-[N,N-(dimethylaminoethoxy)]-2-phenyl-1,7,7-trimethylbicyclo[2.2.1]heptane or a pharmaceutically acceptable acid addition salt thereof and as component B) morphine, an opioide type analgesic and/or a non-opioide type analgesic with inert pharmaceutically acceptable carriers and/or auxiliary agents and bringing the mixture into a galenic form.

10. Use of a combination comprising as component A) (1R,2S,4R)-(−)-2-[N,N-(dimethylaminoethoxy)]-2-phenyl-1,7,7-trimethylbicyclo[2.2.1]heptane or a pharmaceutically acceptable acid addition salt thereof and as component B) morphine, an opioide type analgesic and/or a non-opioide type analgesic for the alleviation of pain.

11. Use of a combination comprising as component A) (1R,2S,4R)-(−)-2-[N,N-(dimethylaminoethoxy)]-2-phenyl-1,7,7-trimethylbicyclo[2.2.1]heptane or a pharmaceutically acceptable acid addition salt thereof and as component B) morphine, an opioide type analgesic and/or a non-opioide type analgesic for the preparation of analgesic pharmaceutical compositions.

12. Process for the alleviation of pain which comprises administering to the patient in need of such treatment a pharmaceutically active dose of a combination comprising as component A) (1R,2S,4R)-(−)-2-[N,N-(dimethylaminoethoxy)]-2-phenyl-1,7,7-trimethylbicyclo[2.2.1]heptane or a pharmaceutically acceptable acid addition salt thereof and as component B) morphine, an opioide type analgesic and/or a non-opioide type analgesic.

13. Use of (1R,2S,4R)-(−)-2-[N,N-(dimethylaminoethoxy)]-2-phenyl-1,7,7-trimethylbicyclo[2.2.1]heptane or a pharmaceutically acceptable acid addition salt thereof to increase the effect of morphine.
